(19) United States
(12) Patent Application Publication
(10) Pub. No.: US 2015/0216462 A1
(43) Pub. Date: Aug. 6, 2015

(54) Analyte Sensor

(71) Applicant: Abbott Diabetes Care Inc., Alameda, CA (US)

(72) Inventors: Kevin Thomas Kastanos, San Lorenzo, CA (US); Gary Ashley Stafford, Hayward, CA (US); Craig V. Murray, Coeur d'Alene, ID (US)

(73) Assignee: Abbott Diabetes Care Inc., Alameda, CA (US)

(21) Appl. No.: 14/687,523

(22) Filed: Apr. 15, 2015

Related U.S. Application Data
(63) Continuation of application No. 12/870,818, filed on Aug. 28, 2010.
(60) Provisional application No. 61/238,159, filed on Aug. 29, 2009.

(51) Int. Cl. A61B 5/1486; A61B 17/34

(52) U.S. Cl. CPC A61B 5/14865; A61B 17/3468; A61B 2017/00004

(57) ABSTRACT

An analyte sensor configured to be implanted in the body of the user unassisted by an introducer sharp.

### RELATED APPLICATION

The present application is a continuation of U.S. patent application Ser. No. 12/870,818 filed Aug. 28, 2010, which claims priority under §35 U.S.C. 119(e) to U.S. Provisional Application No. 61/238,159 filed Aug. 29, 2009, entitled “An Insertable Analyte Sensor-Sharp”, the disclosure of each of which are incorporated by reference for all purposes.

### FIELD OF THE DISCLOSURE

The present disclosure is generally directed to analyte sensors. More particularly, the disclosure is directed to analyte sensors that are capable of insertion into the body of a user unassisted by an introducer sharp.

### BACKGROUND

Diabetes Mellitus is an incurable chronic disease in which the body does not produce or properly utilize insulin. Insulin is a hormone produced by the pancreas that regulates blood sugar (glucose). In particular, when blood sugar levels rise, e.g., after a meal, insulin lowers the blood sugar levels by facilitating blood glucose to move from the blood into the body cells. Thus, when the pancreas does not produce sufficient insulin (a condition known as Type I Diabetes) or does not properly utilize insulin (a condition known as Type II Diabetes), the blood glucose remains in the blood resulting in hyperglycemia or abnormally high blood sugar levels.

The vast and uncontrolled fluctuations in blood glucose levels in people suffering from diabetes can cause long-term, serious complications. Some of these complications include blindness, kidney failure, and nerve damage. Additionally, it is known that diabetes is a factor in accelerating cardiovascular diseases such as atherosclerosis (hardening of the arteries), leading to stroke, coronary heart disease, and other diseases. Accordingly, one important and universal strategy in managing diabetes is to control blood glucose levels.

The first step in managing blood glucose levels is testing and monitoring blood glucose levels by using conventional techniques, such as drawing blood samples, applying the blood to a test strip, and determining the blood glucose level using colorimetric, electrochemical, or photometric test meters. Another more recent technique for monitoring blood glucose levels is by using a continuous or automatic glucose monitoring system, such as for example, the FreeStyle Navigator® continuous glucose monitoring system manufactured by Abbott Diabetes Care Inc. Unlike in vitro blood glucose meters, continuous analyte monitoring systems employ an insertable or implantable sensor, which continuously detects and monitors glucose levels.

In such systems, implantable sensors are generally at least partially inserted into the body of a user with an insertion device. Typically, the insertion device includes an introducer sharp, e.g., metal sharp, associated with the sensor which is used to puncture the user's skin. In some instances, the metal sharp is coupled to the portion of the sensor that is configured for implantation. To that end, the introducer sharp and sensor are driven into the skin of a user simultaneously or in tandem. In this manner, the introducer sharp pierces the skin to establish an entry path or point for the sensor to the internal environment and biological fluid that is sampled for analyte concentration levels. Some drawbacks to the typical method of analyte sensor insertion include not just the trauma to the skin during the piercing of tissue, but also the occurrence of early signal attenuation ((ESA) where sensor signals are attenuated typically during the initial time period (for example, during the first 1-10 hours) measured from sensor insertion) associated with the conventional implantation techniques using an introducer sharp. Presently, there exists a need to insert or implant an analyte sensor with improved comfort and operation.

### INCORPORATED BY REFERENCE

The following patents, applications and/or publications are incorporated herein by reference for all purposes: U.S. Pat. Nos. 4,545,382; 4,711,245; 5,262,035; 5,262,305; 5,264,104; 5,320,715; 5,356,786; 5,509,410; 5,543,326; 5,593,852; 5,601,435; 5,628,890; 5,820,551; 5,822,715; 5,899,855; 5,918,603; 6,071,391; 6,103,033; 6,120,676; 6,121,009; 6,134,461; 6,143,164; 6,144,837; 6,161,095; 6,175,752; 6,270,455; 6,284,478; 6,299,757; 6,338,790; 6,377,894; 6,461,496; 6,503,381; 6,514,460; 6,514,718; 6,540,891; 6,560,471; 6,579,690; 6,591,125; 6,592,745; 6,600,997; 6,605,200; 6,605,201; 6,616,819; 6,618,934; 6,650,471; 6,654,625; 6,676,816; 6,730,200; 6,736,957; 6,746,582; 6,749,740; 6,764,581; 6,773,671; 6,881,551; 6,893,545; 6,932,892; 6,932,894; 6,942,518; 7,041,468; 7,167,818; and 7,299,082; U.S. Published Application Nos. 2004/0186365, now U.S. Pat. No. 7,811,231; 2005/0182306, now U.S. Pat. No. 8,771,183; 2006/0025662, now U.S. Pat. No. 7,740,581; 2006/0091006; 2007/0056858, now U.S. Pat. No. 8,298,389; 2007/0068807, now U.S. Pat. No. 7,846,311; 2007/0095661; 2007/0108048, now U.S. Pat. No. 7,918,975; 2007/0199818, now U.S. Pat. No. 7,811,430; 2007/0227911, now U.S. Pat. No. 7,887,682; 2007/0233013; 2008/0066305, now U.S. Pat. No. 7,895,740; 2008/0081977, now U.S. Pat. No. 7,618,369; 2008/0102441, now U.S. Pat. No. 7,822,557; 2008/0148873, now U.S. Pat. No. 7,802,467; 2008/0161666; 2008/0267823; and 2009/0054748, now U.S. Pat. No. 7,885,698; U.S. patent application Ser. No. 11/461,725, now U.S. Pat. No. 7,866,026; Ser. Nos. 12/131,012; 12/393,921, 12/242,823, now U.S. Pat. No. 8,219,173; Ser. No. 12/363,712, now U.S. Pat. No. 8,346,335; Ser. Nos. 12/495,709; 12/698,124; 12/698,129; 12/714,439; 12/794,721, now U.S. Pat. No. 8,595,607; and Ser. No. 12/842,013, and U.S. Provisional Application Nos. 61/317,243, 61/345,562, and 61/361,374.

### SUMMARY

The present disclosure provides analyte sensors configured to detect an analyte of interest in a biological fluid. An exemplary analyte sensor includes a body having proximal portion, a distal end portion, and first and second surfaces defined between the proximal and distal end portions. In certain embodiments, analyte sensors include a distal tail section extending downwardly from the proximal portion of the body. Additionally, some embodiments also include analyte sensors that include a distal end including a downward taper thereby defining a pointed or substantially pointed tip. The tip may be configured to pierce through one or more layers of skin for at least partial insertion of the sensor in the body of the user.

In certain embodiments, the body of the analyte sensors can be formed from a substrate, such as, for example, a flexible material. In certain embodiments, at least the tip includes a polymer coating. In this regard, the polymer coating provides rigidity to the tip of the sensors to render the tip capable of piercing the skin of a user. In certain embodiments, the distal tip can be implanted into the skin of the user unassisted by an introducer sharp, such as a metal sharp for example, which is typically part of sensor insertion kits.

In certain embodiments, the polymer coating can be applied to at least one or both opposing lateral sides of the sensor body, thereby providing additional rigidity to the sensor body for insertion through the skin of the user.

In certain embodiments, a plurality of polymers is applied to the sensor body. For example, the plurality of polymers may include polymers having different durometers. In certain embodiments, the application of a plurality of polymers to the sensor body defines a sensor body having a varied or variable flexibility across or along a length of the sensor body. Certain embodiments may include only one polymer applied in multiple layers along a length or surface of the sensor body. In other embodiments, multiple layers define or result in a varied or variable thickness along a surface of the sensor body.

In certain embodiments, the polymer applied to the sensor body may include a biocompatible or bioresorbable polymer. For example, the polymer can be a lactic acid polymer, such as polylactide (PLA) or a derivative thereof. In certain embodiments, at least some of the polymer coating dissolves in the body of the user during a period of implantation of the sensor.

Various coating methods can be employed to apply the polymer to the sensor body. For the purpose of illustration, some non-limiting techniques include spin coating, drip coating, dip coating, spray coating, direct coating or immersion. However, other techniques common to the application of coatings on medical devices can be used.

In another aspect, the distal tip of the sensor can be formed from a rigid polymer to render the tip capable of piercing the skin of the user and implanting at least a portion of the sensor in the body of the user. In certain embodiments, the polymer dissolves in the body of the user during a period of implantation of the sensor. Accordingly, the rigidity or degree of rigidity of the distal tip can be temporal, such that the sensor body regains its flexibility after the tip dissolves away from the sensor body.

In certain embodiments, sensors may include one or more protrusions extending from one or more surfaces of the sensor body. The protrusion can be configured to engage a carrier device. In certain embodiments, the carrier device can have a body including at least one flange along a surface. For example, the carrier device can include first and second opposing flanges disposed at lateral opposing ends of the carrier body. In this manner, the one or more protrusions extending from the surface of the sensor body can engage at least one flange of the carrier body. The carrier device can be used to insert the sensor into the skin of the user, for example. For instance, the carrier device in some instances is part of an inserter device.

In certain embodiments, analyte sensors can have a unitary structure. In certain embodiments, the sensor body can be formed from injection molded techniques. In particular, on embodiment includes a body that can be formed from a micro-molding technique.

In certain embodiments, at least the distal tip of the sensor body can include a lubricious coating, such as polytetrafluoroethylene (PTFE), for example.

In certain embodiments, analyte sensors may include one or more electrodes that are disposed on the distal portion including a working electrode, and a sensing layer disposed on the working electrode.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a block diagram of an analyte monitoring system for use in one or more embodiments of the present disclosure;

FIG. 2A illustrates an exemplary analyte sensor in one or more embodiments of the present disclosure;

FIG. 2B illustrates a cross-sectional view of the analyte sensor of FIG. 2A;

FIG. 3 illustrates an analyte sensor having a tapered distal tip in accordance with one embodiment of the present disclosure;

FIGS. 4A-4D illustrate an analyte sensor including a dimple or protrusion and associated insertion assembly carrier for use in one or more embodiments of the present disclosure;

FIG. 5 is a flow chart illustrating a method of forming an analyte sensor in certain embodiments of the present disclosure; and

FIG. 6 is a flow chart illustrating a method of inserting an analyte sensor in certain embodiments of the present disclosure.

### DETAILED DESCRIPTION

Before the present disclosure is described in detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges as also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

Various exemplary embodiments of the analyte monitoring system and methods of the disclosure are described in further detail below. Although the disclosure is described primarily with respect to a glucose monitoring system, each aspect of the disclosure is not intended to be limited to the particular embodiment so described. Accordingly, it is to be understood that such description should not be construed to limit the scope of the disclosure, and it is to be understood that the analyte monitoring system can be configured to monitor a variety of analytes, as described below.

Embodiments of the present disclosure are directed to analyte sensors for use with an analyte monitoring system such as, for example, a continuous, semi-continuous, or a discrete glucose monitoring system. Embodiments include analyte sensors for transcutaneous or subcutaneous placement and configured to facilitate insertion through a skin layer of a user. In certain embodiments, the analyte sensors, in conjunction with sensor electronics, monitor the level of an analyte in a user and transmit data associated with the monitored analyte level to a receiver unit.

Embodiments of the subject disclosure are described primarily with respect to glucose monitoring devices and systems, and methods of glucose level monitoring, for convenience only, and such description is in no way intended to limit the scope of the disclosure. It is to be understood that the analyte monitoring system may be configured to monitor a variety of analytes at the same time or at different times.

For example, analytes that may be monitored include, but are not limited to, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times, with a single sensor or with a plurality of sensors which may use the same on body electronics (e.g., simultaneously) or with different on body electronics.

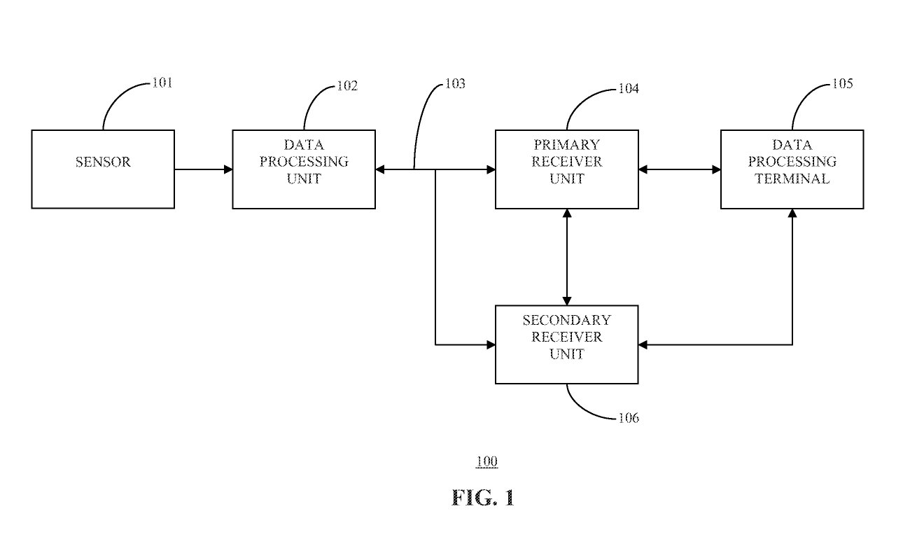

In certain embodiments of the present disclosure, as illustrated in FIG. 1, an analyte monitoring system 100 includes a sensor 101, a data processing unit (e.g., sensor electronics) 102 connectable to sensor 101, and a primary receiver unit 104 which is configured to communicate with the data processing unit 102 via a communication link 103. In certain embodiments, sensor 101 is disposed after a useful sensor life, e.g. 3 days or 7 days or 14 days or more, while data processing unit 102 is reusable and can be used with multiple sensors. In other aspects of the present disclosure, sensor 101 and data processing unit 102 may be configured as a single integrated assembly, whereby data processing unit 102 is discarded or replaced along with sensor 101 after the useful sensor life of sensor 101.

In certain embodiments, primary receiver unit 104 may be further configured to transmit data to a data processing terminal 105 to evaluate or otherwise process or format data received by primary receiver unit 104. Data processing terminal 105 may be configured to receive data directly from data processing unit 102 via a communication link which may optionally be configured for bi-directional communication. Further, data processing unit 102 may include one or more transmitters or one or more transceivers, to transmit and/or to receive data to and/or from primary receiver unit 104, data processing terminal 105, or a secondary receiver unit 106. Description of the various components of analyte monitoring systems can be found in, among others, U.S. Pat. No. 6,175,752 and U.S. patent application Ser. No. 12/143,731, now U.S. Pat. No. 8,597,188, Ser. No. 12/143,734, now U.S. Pat. No. 8,617,069 and Ser. No. 12/698,124, disclosures of each of which are incorporated by reference herein in their entirety for all purposes.

In certain embodiments, analyte sensors of the analyte monitoring system (FIG. 1) are configured to detect and monitor an analyte of interest in a biological sample of a user. The biological sample can be a biological fluid containing the analyte of interest, such as (but not limited to) interstitial fluid, blood, and urine. During use, in certain embodiments, analyte sensors are physically positioned in or on the body of a user whose analyte level is being monitored and can be configured to generate signals related to the analyte level of the user and convert the generated signals into a corresponding data signal for transmission by the transmitter. In certain embodiments, analyte sensors of the subject disclosure are implantable into a subject's body for a period of time (e.g., three to ten days or more) to contact and monitor an analyte level present in the biological fluid. In this regard, sensors of the subject disclosure can be disposed in the subject's body at a variety of sites, including intramuscularly, transcutaneously, intravascularly, or in a body cavity.

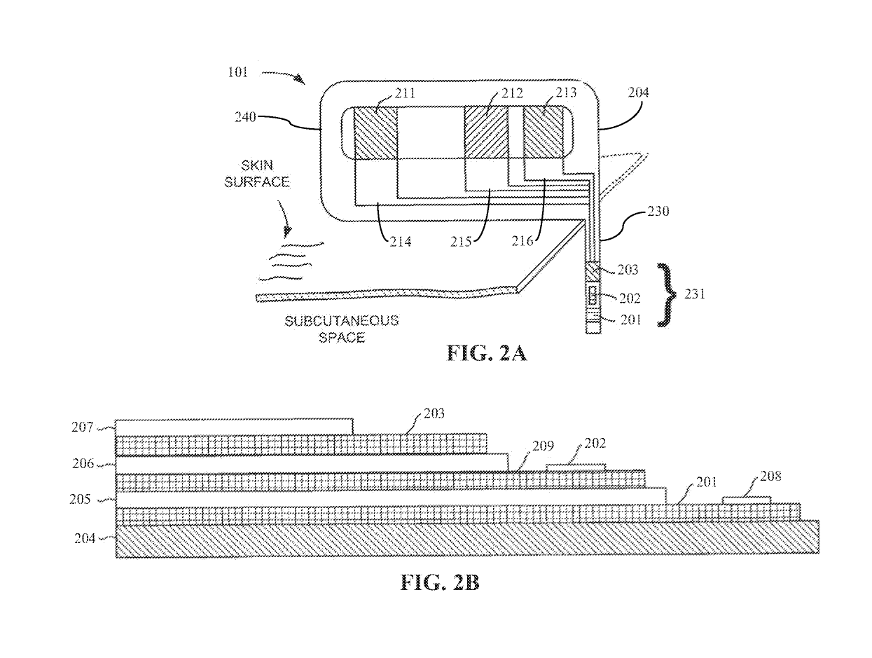

FIG. 2A illustrates an exemplary analyte sensor, such as analyte sensor 101 of the analyte monitoring system 100 of FIG. 1. Generally, analyte sensor 101 can include a body formed from a substrate 204 and one or more electrodes 201, 202, 203 formed on the sensor body, as shown in FIG. 2A. As further illustrated in FIG. 2A, the sensor can include conductive traces 214, 215, and 216 extending from electrodes 201, 202, and 203 to corresponding respective contacts 211, 212, 213 to define electrical contacts to electrically couple with data processing unit 102 (FIG. 1) circuitry.

In certain embodiments, sensor 101 is at least partially implanted into a user's body for a period of days, e.g., 3 days to 7 days to 14 days or more. The substrate 204 can be formed from a relatively flexible material to improve comfort for the user and reduce damage to the surrounding tissue of the insertion site. Suitable materials for a flexible substrate include, for example, non-conducting plastic or polymeric materials and other non-conducting, flexible, deformable materials. Suitable plastic or polymeric materials include thermoplastics such as polycarbonates, polyesters (e.g., Mylar® and polyethylene terephthalate (PET)), polyvinyl chloride (PVC), polyurethanes, polyethers, polyamides, polyimides, or copolymers of these thermoplastics, such as PETG (glycol-modified polyethylene terephthalate).

In other embodiments, analyte sensor 101 can include substrate 204 at least partially formed from a relatively rigid material. Some suitable non-limiting examples of rigid materials that may be used to form the substrate include poorly conducting ceramics, such as aluminum oxide and silicon dioxide. Further, substrate 204 can be formed from an insulating material. Suitable insulating materials include polyurethane, teflon (fluorinated polymers), polyethyleneterephthalate (PET, Dacron) or polyimide.

In certain embodiments, as illustrated in FIG. 2A, analyte sensor 101 can be configured to include a distal end 230, which includes distal tip 231, a proximal portion 240, and opposing lateral sides. In certain embodiments, the proximal portion 240 and distal end 230 can have different widths. For example, the distal end 230 of the sensor 101 may have a relatively narrow width, while the proximal portion 240 of the sensor 101 may have a relatively wide width. During use, at least a portion of the distal end 230, including the distal tip 231, can be implanted under the skin surface in the subcutaneous space of the user, while the proximal portion 240 of the sensor extends exterior to the user's body. For example, in certain embodiments, insertable sensors designed for continuous or periodic monitoring of an analyte during normal activities of the user, can include a distal end 230 configured to be implanted into the user including a width of 2 mm or less, preferably 1 mm or less, and more preferably 0.5 mm or less in order to minimize discomfort associated with the inserted sensor distal end 230.

Referring now to FIG. 2B, a cross sectional view of the sensor 101 in certain embodiments is shown. In particular, it can be seen that the various electrodes of the sensor 101 as well as the substrate and the dielectric layers are provided in a stacked or layered configuration or construction. For example, as shown in FIG. 2B, in one aspect, sensor 101 includes substrate layer 204, and first conducting layer 201 such as a carbon or gold trace disposed on at least a portion of substrate layer 204, and which may comprise the working electrode. Also shown disposed on at least a portion of first conducting layer 201 is a sensing layer 208.

Referring back to FIG. 2B, a first insulation layer such as first dielectric layer 205 is disposed or stacked on at least a portion of first conducting layer 201, and further, second conducting layer 209, such as another carbon or gold trace, may be disposed or stacked on top of at least a portion of first insulation layer (or dielectric layer) 205. As shown in FIG. 2B, second conducting layer 209 may comprise reference electrode 202, and in one aspect, may include a layer of silver/silver chloride (Ag/AgCl). In certain embodiments, the reference electrode 202 or contact may be integrally formed with second conducting layer 209 such that second conducting layer 209 includes a reference electrode.

Referring still again to FIG. 2B, a second insulation layer 206, such as a dielectric layer, in certain embodiments, may be disposed or stacked on at least a portion of second conducting layer 209. Further, a third conducting layer 203, which may include a carbon or gold trace and that may comprise counter electrode 203, may, in certain embodiments, be disposed on at least a portion of second insulation layer 206. Finally, a third insulation layer 207 may be disposed or stacked on at least a portion of third conducting layer 203. In this manner, the sensor 101 may be configured in a stacked or layered construction or configuration such that at least a portion of each of the conducting layers is separated by a respective insulation layer (for example, a dielectric layer).

In certain embodiments, analyte sensor 101 can include a sensing layer and a barrier layer disposed on the substrate. Sensing layer includes one or more components designed to facilitate the electrolysis of the analyte of interest, such as for example a catalyst and an electron transfer agent. The components, for example, may be immobilized on the working electrode. Alternatively, the components of the sensing layer may be immobilized within or between one or more membranes or films disposed over the working electrode or the components may be immobilized in a polymeric or sol-gel matrix. Examples of immobilized sensing layers are described in U.S. Pat. Nos. 5,262,035, 5,264,104, 5,264,105, 5,320,725, 5,593,852, 5,665,222, 6,175,752 and 6,990,366, each of which is incorporated herein by reference.

In general, electron transfer agents suitable for use in the invention have structures or charges which prevent or substantially reduce the diffusional loss of the electron transfer agent during the period of time that the sample is being analyzed. The preferred electron transfer agents include a redox species bound to a polymer which can in turn be immobilized on the working electrode. The bond between the redox species and the polymer may be covalent, coordinative, or ionic. Useful electron transfer agents and methods for producing them are described in U.S. Pat. Nos. 5,264,104, 5,356,786, 5,262,035, and 5,320,725 which are incorporated herein by reference.

In certain embodiments, an analyte can be electrolyzed by applying a potential (versus a reference potential) across the working and counter electrodes. When a potential is applied between the working electrode and the counter electrode, an electrical current will flow. The current is a result of the electrolysis of the analyte or a second compound whose level is affected by the analyte. In certain embodiments, the electrochemical reaction occurs via an electron transfer agent and the optional catalyst. Many analytes are oxidized (or reduced) to products by an electron transfer agent species in the presence of an appropriate catalyst (e.g., an enzyme). The electron transfer agent is then oxidized (or reduced) at the electrode. Electrons are collected by (or removed from) the electrode and the resulting current is measured.

As an example, an electrochemical sensor may be based on the reaction of a glucose molecule with two non-leachable ferricyanide anions in the presence of glucose oxidase to produce two non-leachable ferrocyanide anions, two hydrogen ions, and gluconolactone. The amount of glucose present is assayed by electrooxidizing the non-leachable ferrocyanide anions to non-leachable ferricyanide anions and measuring the current. Changes in the concentration of the reactant compound, as indicated by the signal at the working electrode, correspond inversely to changes in the analyte (i.e., as the level of analyte increases then the level of reactant compound and the signal at the electrode decreases).

In certain embodiments, sensing layer components can include, for example, a catalyst to catalyze a reaction of the analyte at the working electrode, or an electron transfer agent to indirectly or directly transfer electrons between the analyte and the working electrode, or both. The catalyst is capable of catalyzing a reaction of the analyte. The catalyst may also, in certain embodiments, act as an electron transfer agent. One example of a suitable catalyst is an enzyme which catalyzes a reaction of the analyte. For example, a catalyst, such as a glucose oxidase, glucose dehydrogenase (e.g., pyrroloquinoline quinone glucose dehydrogenase (PQQ)), or oligosaccharide dehydrogenase, may be used when the analyte is glucose. A lactate oxidase or lactate dehydrogenase may be used when the analyte is lactate. Lactase may be used when the analyte is oxygen or when oxygen is generated or consumed in response to a reaction of the analyte. In other embodiments, the sensing layer can include a peroxidase enzyme and an electron transfer agent to generate a signal at the electrode in response to the hydrogen peroxide. The level of hydrogen peroxide indicated by the sensor then correlates to the level of glucose or lactate. Another sensor which operates similarly can be made using a single sensing layer with both the glucose or lactate oxidase and the peroxidase being deposited in the single sensing layer. Examples of such sensors are described in U.S. Pat. Nos. 5,593,852, and 5,665,222, each of which is incorporated herein by reference.

Preferably, the catalyst is non-leachably disposed on the sensor, whether the catalyst is part of a solid sensing layer in the sensor or solvated in a fluid within the sensing layer. More preferably, the catalyst is immobilized within the sensor (e.g., on the electrode and/or within or between a membrane or film) to prevent unwanted leaching of the catalyst away from the working electrode and into the user. This may be accomplished, for example, by attaching the catalyst to a polymer, cross linking the catalyst with another electron transfer agent (which, as described above, can be polymeric), and/or providing one or more barrier membranes or films with pore sizes smaller than the catalyst.

In many embodiments, the sensing layer contains one or more electron transfer agents in contact with the conductive material of the working electrode. In these embodiments, preferably, at least 90%, more preferably, at least 95%, and, most preferably, at least 99%, of the electron transfer agent remains disposed on the sensor after immersion in the analyte-containing fluid for 24 hours, and, more preferably, for 72 hours. In particular, for an implantable sensor, preferably, at least 90%, more preferably, at least 95%, and most preferably, at least 99%, of the electron transfer agent remains disposed on the sensor after immersion in the body fluid at 37° C. for 24 hours, and, more preferably, for 72 hours.

Like the catalyst, the electron transfer agent can be immobilized on the working electrode. Suitable immobilization techniques include, for example, a polymeric or sol-gel immobilization technique. Alternatively, the electron transfer agent may be chemically (e.g., ionically, covalently, or coordinatively) bound to the working electrode, either directly or indirectly through another molecule, such as a polymer, that is in turn bound to the working electrode.

The electron transfer agent can mediate the transfer of electrons to electrooxidize or electroreduce an analyte and thereby permits a current flow between the working electrode and the counter electrode via the analyte. The mediation of the electron transfer agent can facilitate the electrochemical analysis of analytes which are not suited for direct electrochemical reaction on an electrode. Suitable electron transfer agents include electroreducible and electrooxidizable ions or molecules having redox potentials that are a few hundred millivolts above or below the redox potential of the standard calomel electrode (SCE). Preferably, the electron transfer agents are not more reducing than about −150 mV and not more oxidizing than about +400 mV versus SCE.

Useful electron transfer agents and methods for producing them are described in U.S. Pat. Nos. 5,264,104; 5,356,786; 5,262,035; and 5,320,725, incorporated herein by reference. Although any organic or organometallic redox species can be bound to a polymer and used as an electron transfer agent, the preferred redox species is a transition metal compound or complex. The preferred transition metal compounds or complexes include osmium, ruthenium, iron, and cobalt compounds or complexes. The most preferred are osmium compounds and complexes. It will be recognized that many of the redox species described below may also be used, typically without a polymeric component, as electron transfer agents in a carrier fluid or in a sensing layer of a sensor where leaching of the electron transfer agent is acceptable.

In certain embodiments of the invention, suitable non-releasable electron transfer agents include a redox species coordinatively bound to a polymer. For example, the mediator may be formed by coordination of an osmium or cobalt 2,2′-bipyridyl complex to poly(1-vinyl imidazole) or poly(4-vinyl pyridine). The preferred electron transfer agents are osmium transition metal complexes with one or more ligands, each ligand having a nitrogen-containing heterocycle such as 2,2′-bipyridine, 1,10-phenanthroline, or derivatives thereof. Furthermore, the preferred electron transfer agents also have one or more ligands covalently bound in a polymer, each ligand having at least one nitrogen-containing heterocycle, such as pyridine, imidazole, or derivatives thereof. These preferred electron transfer agents exchange electrons rapidly between each other and the working electrodes so that the complex can be rapidly oxidized and reduced.

One example of a particularly useful electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Preferred derivatives of 2,2′-bipyridine for complexation with the osmium cation are 4,4′-dimethyl-2,2′-bipyridine and mono-, di-, and polyalkoxy-2,2′-bipyridines, such as 4,4′-dimethoxy-2,2′-bipyridine. Preferred derivatives of 1,10-phenanthroline for complexation with the osmium cation are 4,7-dimethyl-1,10-phenanthroline and mono, di-, and polyalkoxy-1,10-phenanthrolines, such as 4,7-dimethoxy-1,10-phenanthroline. Preferred polymers for complexation with the osmium cation include polymers and copolymers of poly(1-vinyl imidazole) (referred to as “PVI”) and poly(4-vinyl pyridine) (referred to as “PVP”). Suitable copolymer substituents of poly(1-vinyl imidazole) include acrylonitrile, acrylamide, and substituted or quaternized N-vinyl imidazole. Most preferred are electron transfer agents with osmium complexed to a polymer or copolymer of poly(1-vinyl imidazole).

Non-leachability may be accomplished, for example, by providing barriers (e.g., the electrode, substrate, membranes, and/or films) around the sensing layer which prevent the leaching of the components of the sensing layer. One example of such a barrier layer is a microporous membrane or film which allows diffusion of the analyte into the sensing layer such that contact is made with the components of the sensing layer, but reduces or eliminates the diffusion of the sensing layer components (e.g., an electron transfer agent and/or a catalyst) out of the sensing layer.

In certain embodiments, the sensor includes a barrier layer to act as a diffusion-limiting barrier to reduce the rate of mass transport of the analyte into the region around the working electrode. A steady state concentration of the analyte in the proximity of the working electrode (which is proportional to the concentration of the analyte in the body or sample fluid) can be established by limiting the diffusion of the analyte.

In certain embodiments, the permeability of the analyte through the barrier layer can vary little or not at all with temperature, so as to reduce or eliminate the variation of current with temperature. Accordingly, for this reason, in certain embodiments, in the biologically relevant temperature range from about 25° C. to about 45° C., and most importantly from 30° C. to 40° C., neither the size of the pores in the film nor its hydration or swelling change excessively. The barrier layer can be made using a film that absorbs less than 5 wt. % of fluid over 24 hours. For implantable sensors, the barrier layer can be made using a film that absorbs less than 5 wt. % of fluid over 24 hours at 37° C.

Suitable materials for the barrier layer include membranes that do not swell in the analyte-containing fluid that the sensor tests. Suitable membranes include 3 nm to 20,000 nm diameter pores. For example, membranes may include 5 nm to 500 nm diameter pores with well-defined, uniform pore sizes and high aspect ratios. For example, the aspect ratio of the pores can be two or greater, e.g., five or greater. The permeability of the barrier layer membrane can be configured such that changes no more than 4% in the range from 30° C. to 40° C. occur when the membranes reside in the subcutaneous interstitial fluid.

In certain embodiments, the barrier layer can also limit the flow of oxygen into the sensor, thereby improving the stability of sensors that are used in situations where variation in the partial pressure of oxygen causes non-linearity in sensor response. In these embodiments, the barrier layer restricts oxygen transport by at least 40%, preferably at least 60%, and more preferably at least 80%, than the membrane restricts transport of the analyte. For a given type of polymer, films having a greater density (e.g., a density closer to that of the crystalline polymer) are preferred. Polyesters, such as polyethylene terephthalate, are typically less permeable to oxygen and are, therefore, preferred over polycarbonate membranes.

Referring back to the Figures, in accordance with one aspect of the present disclosure, some embodiments of sensor 101 can be configured for insertion or implantation in a user's body without the need to use an introducer sharp. Accordingly, in certain embodiments, the distal end 230 of sensor 101 is configured for insertion into the body of a user unassisted by an introducer or a metal sharp to pierce the tissue at the site of insertion. Elimination of the need for a conventional metal sharp to facilitate or effect insertion of the sensor 101 can provide insertion in a single motion and further, improve user comfort and operation, in addition to more effectively addressing ESA condition sometimes associated with implantation of sensors. Further description of ESA condition associated with transcutaneously positioned analyte sensors are provided in, among others, U.S. patent application Ser. No. 11/925,689, now U.S. Pat. No. 8,135,548, Ser. No. 12/362,475, now U.S. Pat. No. 8,103,456, and Ser. No. 12/363,712, now U.S. Pat. No. 8,346,335, the disclosures of each of which are incorporated herein by reference for all purposes.

In certain embodiments, analyte sensor 101 may include distal end 230 with distal tip 231 configured to have a reduced size as compared to an introducer sharp, which typically has size, e.g., circumference, sufficient to hold the sensor distal end 230. As such, the size of the wound created by the sensor insertion without the use of an introducer sharp can be much smaller than a wound created by a conventional metal sharp. Further, in certain embodiments, the reduced blood flow and/or blood clotting associated with insertion without the use of an introducer sharp may, thereby lower or minimize occurrence of ESA conditions. Additionally, elimination of the metal sharp and the extra parts needed to operate the sharp not only reduces required components for an inserter device, e.g. a retraction spring in an inserter device, but also can facilitate an automated manufacturing process, thereby reducing manufacturing costs.

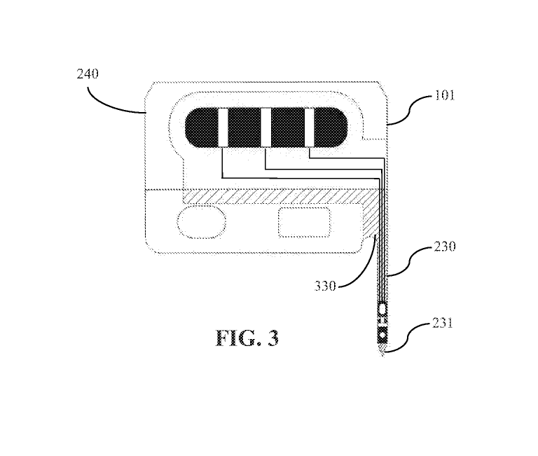

FIG. 3 illustrates an analyte sensor having a tapered distal tip in accordance with certain embodiments of the present disclosure. Referring to FIG. 3, distal tip 231 of distal end 230 of analyte sensor 101 includes a downward gradual taper to define a pointed tip. In this manner, the distal tip 231 can be modified for skin piercing capability. To this end, in certain embodiments, at least a portion of the distal tip 231 of the distal end 230 of the sensor 101 can be coated with a polymer suitable to render at least the pointed distal tip 231 rigid. In certain embodiments, the analyte sensor 101 may include a coated portion 330 (denoted by the hatch marking), which may be covered or coated with the polymer coating described above to provide sufficient rigidity for sensor insertion without the use of an introducer sharp. In other embodiments, additional or alternative areas of the sensor may be coated with the polymer coating to provide sensor rigidity. In certain embodiments, only the distal tip 231 is coated with the polymer coating. In this manner, the imputed rigidity by application of the polymer coating to the distal tip 231 can be sufficient to render the distal tip 231 capable of piercing the skin of a user for implantation of at least a portion of the distal end 230 of the sensor body, unassisted by a sharp.

In certain embodiments, first and second opposing lateral ends of the sensors can also be coated with one or more polymers to provide additional rigidity to the sensors for ease of insertion into the skin surface.

Various suitable rigid polymers can be employed in coating portions of the sensor 101. Some non-limiting examples include polyamide, polycarbonates, polyethylenes, polyurethanes, Kevlar®, block copolymers, and cross-linked polymers. Additionally, the coating may include polymer blends, such as a polylactic acid blends, polyamide blends, and the like. In still other embodiments, the coating may include lactic acid polymer or polylactide (PLA) and various synthesized polymerized forms including poly-DL-lactide (PDLLA) and poly-L-lactide (PLLA). In certain embodiments, the polymer coating includes one or more biocompatible polymers. In certain embodiments, the polymer one or more bioresorbable polymers.

In certain embodiments, a plurality of polymer layers can be applied to the sensor. In this regard, one or more polymers can be used. As such the plurality of polymer coatings results in the sensor rigidity, rather than the polymer itself. In certain embodiments, the polymers can be applied to areas in addition to the distal tip. For example, a sensor having a varied flexibility along its longitudinal axis can be formed by coating polymers having different durometers at different sections of the substrate to define a sensor having a varied or variable thickness across a length. In this manner, the sensor body can be configured to have different flexibilities depending upon the number of polymer coats at a section of the sensor substrate.

In certain embodiments, the polymer coating may include a bio-absorbable lactic acid polymer. The bio-absorbable lactic acid polymer applied to the sensor 101, for example to the distal end 230 and the distal tip 231 of the sensor 101, results in rigid sensor body, for example, rigidity to the sensor distal end 230 to facilitate puncture of a skin layer of a user and subsequent insertion of the sensor 101. The rigid sensor allows for insertion without the use of a separate introducer sharp. Further, in certain embodiments, the bio-absorbable lactic acid polymer coating dissolves in the body of the user after insertion. The dissolving of the polymer coating allows for the sensor distal end 230 to regain a level of flexibility in order to minimize the discomfort of the user associated with sensor use.

In certain embodiments, it is desirable to include sections or portions of sensor 101 where no coating is applied. These sections can be masked for protection when the coating is applied to the sensor. For example, any electrodes and/or sensing layers can be masked to avoid unwanted contamination. Further, in certain embodiments, by leaving sections of the sensor uncovered by the coating, the sensor can immediately begin detection of analyte levels without waiting for the bio-absorbable coating to dissolve.

Various coating techniques can be utilized to coat the sensor with a suitable polymer. Some non-limiting techniques include spray coating, spin coating, dip coating, and direct coating techniques. For example, any conventional coating techniques employed in polymer coating cardiovascular stents and other medical devices can be used.

Furthermore, to additionally facilitate insertion and minimize pain to the user, a lubricious coating can be applied at least to the distal tip 231 of sensor 101 to facilitate ease of insertion into the body of a user. In certain embodiments, the lubricous coating reduces friction during insertion. Various lubricious materials can be employed such as, but not limited to, PTFE and high density polyethylene.

In certain embodiments, the distal tapered tip 231 or distal end 230 of the analyte sensor 101 can be formed from a rigid polymer. In this manner, the analyte sensor can include a distal tapered tip 231 configured to pierce the skin of a user without the need for a polymer coating applied to the sensor body. In this manner, analyte sensor 101 with at least the tapered tip 231 can be formed from a polymer or polymer blend sufficiently rigid to pierce a skin layer of a user.

Various techniques can be employed to form the sensor. For example, conventional injection molding techniques can be utilized. Alternatively, a micro-molding technique, such as micro-injection molding, can be employed. The term “micro-molding techniques” includes molding micro-sized parts, larger parts that have miniscule features, and parts made from a fraction of a plastic pellet, for example one weighing fractions of a gram.

Moreover, in certain embodiments, different types of rigid polymers can be employed to form the sensor or at least the distal tail or tip of the sensor by the injection molding or micro-molding techniques. For example, the polymers can include, but are not limited to, polyamides, polyethylenes, polycarbonates, polyurethanes, Kevlar®, acrylnitrile butadiene styrene (“ABS”), ceramic, nylon, glass-filled nylon, and the like. Additionally, the polymer can be a cross-linked polymer or block copolymer. In certain embodiments, the polymer can include a bioresorbable polymer, such as, but not limited to, a lactic acid or polyoxymethylene. In certain embodiments, the bioresorbable polymer can dissolve while implanted into the user's body. After the dissolving of the bioresorbable polymer, the sensor can regain some flexibility.

In the manner described above, in certain embodiments, portion or portions of analyte sensor body is coated or otherwise provided with one or more layers of polymer coating such as, for example, rigid lactic acid polymer. The one or more layers of polymer coating on the sensor increases the rigidity of the sensor such that the sensor is configured for transcutaneous positioning through a skin surface without using a conventional metal sharp or needle in conjunction with a sensor insertion device.

Referring again to the Figures, analyte sensor configurations in certain embodiments include an integrated sensor and sensor introducer configuration, for example, by injection molding the sensor and introducer as a single device, and also including molding, for example, a dimple or depression to the sensor for retaining the sensor in position prior to use.

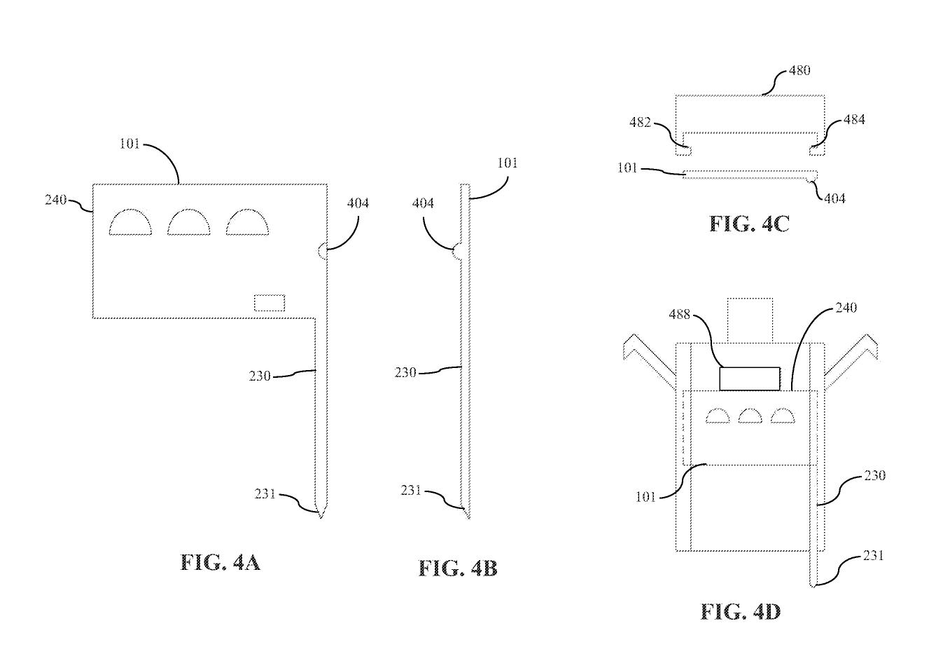

More particularly, FIGS. 4A-4D illustrate an analyte sensor including a dimple or protrusion and associated insertion assembly carrier for use in one or more embodiments of the present disclosure. In certain embodiments, one or more dimples or protrusions 404 are formed in at least one of the surfaces of the sensor substrate 204. The one or more dimples or protrusions 404 can be formed to extend axially from a main surface of the sensor 101, as shown in FIGS. 4A and 4B. In certain embodiments, sensor 101 can be releasably retained within a carrier device 480, as illustrated in FIG. 4C. For example, in certain embodiments, sensor 101 can be configured to have a sliding engagement to carrier device 480. Carrier device 480 can include first and second opposing flanges 482 and 484 to respectively receive opposing first and second lateral sides of sensor 101. Accordingly, as illustrated in FIGS. 4C and 4D, opposing flanges 482, 484 can serve as a nesting area for sensor 101. In this manner, the one or more dimples or protrusions 404 can provide friction engagements with at least one flange to retain sensor 101 within carrier device 480 until deployment into the body of the user. Accordingly, sensor 101 can be held in place by the dimple or protrusion 404. Exemplary sensors and associated insertion devices can be found in, among others, U.S. Pat. No. 7,381,184 and U.S. patent application Ser. No. 11/240,258, the disclosures of each of which are incorporated herein by reference for all purposes.

In certain embodiments, carrier device 480 can include a carrier stop 488 configured to abut the proximal portion 240 of sensor 101. In this manner, carrier stop 488, in certain embodiments, may maintain sensor 101 in position within carrier device 480 and in position without undesirable displacement during deployment of sensor 101.

Exemplary insertion assemblies and description for retention and insertion of analyte sensors can be found in, among others, U.S. Pat. Nos. 6,990,366 and 7,381,184, U.S. patent application Ser. No. 12/698,129, and U.S. Provisional Patent Application Nos. 61/317,243, 61/345,562 and 61/361,374, the disclosures of each of which are incorporated herein by reference for all purposes.

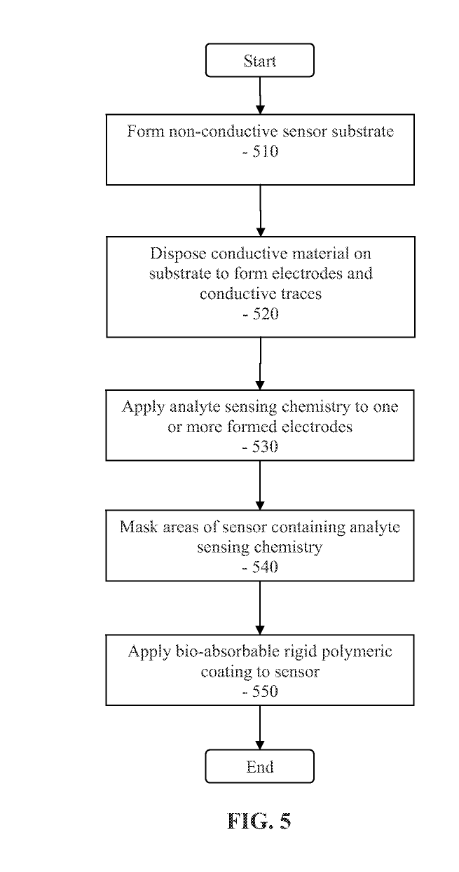

FIG. 5 is a flow chart illustrating a routine for forming a rigid analyte sensor in certain embodiments. Referring to FIG. 5, in certain embodiments, a sensor substrate is formed of a non-conducting material in the desired sensor shape including a pointed distal tip (510). On the formed substrate, conductive material is disposed to form electrodes on the distal end of the sensor, contact pads on the proximal portion of the sensor, and conductive traces connecting the electrodes and corresponding contact pads (520). After formation of the electrodes, analyte sensing chemistry is applied to one or more of the electrodes (530), for example, on a formed working electrode. Areas of the sensor that contain the analyte sensing chemistry are masked (540) and a bio-absorbable rigid polymeric coating, such as the lactic acid polymer coating described above, is applied to at least the distal tip and distal end of the sensor (550), while the masking of the analyte sensing chemistry sections prevents coating of these sections. The formed sensor includes a rigid and sharp distal end to facilitate insertion through the skin layer of a user without the need of a separate introducer sharp.

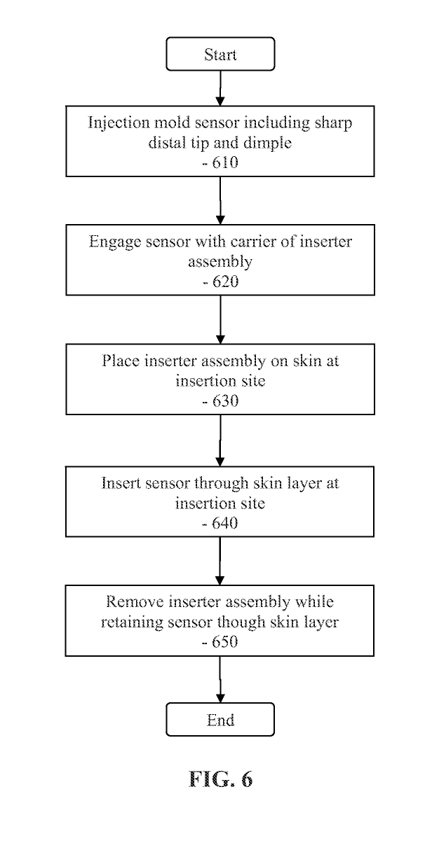

FIG. 6 is a flow chart illustrating a method of inserting an analyte sensor in certain embodiments. Referring to FIG. 6, in certain embodiments, a sensor is formed including a sharp distal tip and dimple or protrusion through techniques including injection molding or micro-injection molding (610). The formed sensor is engaged to a carrier of an inserter assembly (620) with the dimple or protrusion assisting in engagement with the carrier as described above. The inserter assembly with loaded sensor is placed at the insertion site of a user's skin (630) and the inserter assembly is activated and inserts the sensor through the skin (640). The inserter assembly may include an automatic or manual inserter assembly. The sharp distal tip of the sensor facilitates insertion of the sensor through the skin layer of the user without the need for a separate introducer sharp. As such, the inserter assembly may be removed from the insertion site, with the sensor retained within the skin layer (650), without the need for a retraction of an introducer sharp.

The above described methods of applying a rigid polymeric coating to a sensor to facilitate insertion without the use of a separate introducer and forming a sharp sensor tip to facilitate insertion and a dimple or protrusion to facilitate coupling with a carrier of an inserter assembly, using micro-injection molding techniques can be applied to various sensors in addition to the particular size, dimension, and configuration of sensors described herein. Sensors to which these techniques may be applied include sensors described in, but not limited to, U.S. Pat. Nos. 5,593,852, 6,103,033, 6,134,461, 6,175,752, and U.S. patent application Ser. Nos. 12/393,921 and 12/698,124, the disclosures of each of which are incorporated herein by reference for all purposes.

In one aspect, an analyte sensor configured to detect an analyte of interest in a biological fluid may include a body including a proximal portion, a distal end, first and second surfaces defined therebetween, and one or more electrodes disposed on at least one surface, wherein the distal end includes a downward taper to define a substantially pointed tip, and further wherein the substantially pointed tip is configured to pierce through one or more layers of skin to at least partially insert the sensor in the skin of a user.

In certain embodiments, the body of the sensor may be formed from a substrate.

In certain embodiments, at least the tip may include a polymer coating.

In certain embodiments, the polymer coating applied to the tip may provide sufficient rigidity to the tip to pierce the skin of a user.

In certain embodiments, at least the distal tip of the sensor is implantable into the skin of a user without requiring a separate introducer sharp.

In certain embodiments, the polymer coating may be applied to at least one of the opposing lateral sides of the sensor body.

In yet certain embodiments, a plurality of polymers may be applied to the sensor body.

Moreover, in certain embodiments, the plurality of polymers may include polymers having different durometers.

In certain embodiments, the plurality of polymers applied to the sensor substrate may define a sensor having varied flexibility along a length thereof.

In certain embodiments, the plurality of polymers may include one polymer applied in a plurality of layers along a surface of the sensor body.

In a certain embodiments, the plurality of layers may define a varied thickness along a surface of the sensor body.

In certain embodiments, the polymer may include a biocompatible or bioresorbable polymer.

In certain embodiments, the polymer may include a lactic acid polymer.

In certain embodiments, the lactic acid may dissolve in the body of the user during implantation of the sensor.

In certain embodiments, the polymer may include a polyamide, polycarbonate, polyurethane, crosslinked polymer, or block copolymer.

In certain embodiments, the polymer coating may be applied to the sensor body by one or more techniques including spin coating, drip coating, dip coating, spray coating, direct coating or immersion.

In certain embodiments, the distal tip may be formed from a polymer.

In certain embodiments, the sensor may be formed from an injection molded technique.

In certain embodiments, the polymer may have sufficient rigidity to pierce the skin of the user, and further the pierced skin may be sufficient to implant at least part of the sensor in the body of the user.

In certain embodiments, the polymer may be a resorbable polymer.

In certain embodiments, the polymer may include lactic acid or derivative.

In certain embodiments, the polymer includes polylactic acid.

In certain embodiments, the polylactic acid may dissolve in the body of the user during a period of implantation of the sensor.

In certain embodiments, the analyte sensor may include one or more protrusions extending from one or more surfaces of the sensor body.

In certain embodiments, the protrusion may be configured to engage a carrier device.

In certain embodiments, the carrier device may have a body including at least one flange along a surface.

In certain embodiments, the carrier device may include first and second opposing flanges disposed at lateral opposing ends of the carrier body.

In certain embodiments, the one or more protrusions extending from the surface of the sensor body may engage at least one flange of the carrier body.

In certain embodiments, the carrier may include a stop configured to abut the proximal portion of the sensor body during engagement.

In certain embodiments, the sensor may be a unitary structure.

In certain embodiments, the body may be formed from an injection molded technique.

In certain embodiments, the body may be formed from a micro-molding technique.

In certain embodiments, at least the tip may include a lubricious coating.

In certain embodiments, the one or more electrodes may comprise gold.

In a certain embodiments, the gold electrodes may be formed by a laser ablation technique.

Aspects of the present disclosure may include an analyte sensor configured to detect an analyte of interest in a biological fluid, the analyte sensor comprising an injection molded body including a proximal portion, a distal end, and first and second surfaces defined therebetween, and one or more electrodes disposed on the distal end of the body, the one or more electrodes including a working electrode, wherein the distal end includes a downward taper to define a substantially pointed tip, wherein the tip is configured to pierce through one or more layers of skin to at least partially insert the sensor in the skin of a user without the requirement of an introducer sharp.

In certain embodiments, the analyte sensor may comprise a sensing layer.

In certain embodiments, the sensing layer may comprise an electron transfer agent.

In certain embodiments, the electron transfer agent may be a redox mediator.

In certain embodiments, the electron transfer agent may comprise osmium transition metal complexes and one or more ligands.

In certain embodiments, the electron transfer agent may be configured to transfer electrons directly between the analyte and the working electrode.

In yet certain embodiments, the electron transfer agent may be configured to transfer electrons indirectly between the analyte and the working electrode.

In certain embodiments, the sensing layer may comprise a redox polymer.

In certain embodiments, the redox polymer may comprise osmium.

In certain embodiments, the sensing layer may comprise a catalyst.

In certain embodiments, the catalyst may comprise an enzyme.

In certain embodiments, the catalyst may act as an electron transfer agent.

In certain embodiments, the enzyme may comprise glucose oxidase.

In certain embodiments, the enzyme may comprise glucose dehydrogenase.

In certain embodiments, the sensing layer may be configured such that the reaction of glucose in the presence of an enzyme forms hydrogen peroxide.

In certain embodiments, the level of hydrogen peroxide may correlate to the level of glucose.

Various other modifications and alterations in the structure and method of operation of this disclosure will be apparent to those skilled in the art without departing from the scope and spirit of the embodiments of the present disclosure. Although the present disclosure has been described in connection with particular embodiments, it should be understood that the present disclosure as claimed should not be unduly limited to such particular embodiments. It is intended that the following claims define the scope of the present disclosure and that structures and methods within the scope of these claims and their equivalents be covered thereby.

1. (canceled)

2. A sensor device for measuring an analyte concentration in a host, comprising:
a sensor unit comprising a sensor body, at least one electrode, and a membrane covering at least a portion of the at least one electrode, the sensor body having a blunt tip;
a piercing element comprising a material configured to rapidly dissolve upon insertion into a host, the piercing element abutting the sensor tip and configured for piercing tissue; and
a mounting unit spaced from the sensor tip and configured to support the sensor device on an exterior surface of the host's skin.

3. The sensor device of claim 2, wherein the piercing element is secured to the sensor tip.

4. The sensor device of claim 3, wherein the piercing element is adhered to the sensor tip.

5. The sensor device of claim 2, wherein the piercing element comprises a coating that covers at least a portion of the sensor body including the sensor tip.

6. The sensor device of claim 5, wherein the coating comprises a sharp coating tip.

7. The sensor device of claim 2, wherein the material of the piercing element comprises a material configured to suppress wounding.

8. The sensor device of claim 2, wherein the material of the piercing element comprises a material configured to promote rapid wound healing.

9. The sensor device of claim 2, wherein the material of the piercing element comprises at least one material selected from the group consisting of a synthetic polymer or polylactic acid.

10. The sensor device of claim 2, wherein the material of the piercing element biodegrades or dissolves within a first day after insertion into the host.

11. The sensor device of claim 10, wherein the material of the piercing element biodegrades or dissolves within three hours after insertion into the host.

12. The sensor device of claim 2, wherein the piercing element does not extend past the sensor tip in the direction of the mounting unit, or extends only a nominal amount in said direction.

13. The sensor device of claim 2, wherein the piercing element extends past the sensor tip in the direction of the mounting unit, but stops short of the electrode.

14. The sensor device of claim 2, wherein the mounting unit comprises a guiding portion configured to guide insertion of the sensor unit through the host's skin and to support a column strength of the sensor body such that the sensor unit is configured for insertion through the host's skin without substantial buckling.

15. The sensor device of claim 2, wherein the at least one electrode comprises a working electrode and a reference electrode.

16. The sensor device of claim 2, wherein the sensor body further comprises a support member configured to protect the membrane from damage during insertion of the sensor unit.

17. The sensor device of claim 16, wherein the support member is configured to support at least a portion of the at least one electrode.

18. The sensor device of claim 16, wherein the support member is configured to substantially surround the at least one electrode.

19. The sensor device of claim 2, wherein the mounting unit comprises a sensor electronics unit operatively and detachably connected to the sensor body.

20. The sensor device of claim 19, wherein the sensor electronics unit is configured to be located over a sensor insertion site.
